(19) United States
(12) Patent Application Publication
(10) Pub. No.: US 2012/0111727 A1
(43) Pub. Date: May 10, 2012

(54) CAPILLARY SIEVING ELECTROPHORESIS WITH A CATIONIC SURFACTANT FOR SIZE SEPARATION OF PROTEINS

(76) Inventors: Vladislav Dolnik, Palo Alto, CA (US); William A. Gurske, Fremont, CA (US)

(21) Appl. No.: 13/350,793

(22) Filed: Jan. 15, 2012

Related U.S. Application Data
(63) Continuation-in-part of application No. 12/359,345, filed on Jan. 26, 2009.
Application No. 12/359,345 is a continuation of application No. 12/359,345, filed on Jan. 26, 2009.

(51) Int. Cl. C07K 1/26; C09K 3/00

(52) U.S. Cl. 204/453; 204/451; 252/364; 252/183.11

(57) ABSTRACT

Disclosed herein is a method for size separation of proteins by capillary sieving electrophoresis with cationic surfactant, suitable for size separation of proteins with molecular weights in the range between about 14,000 and about 500,000, further a composition of a separation medium and denaturing solution for said method of separation method. In a preferred embodiment, the separation medium comprises a buffer having pH between about 3 and 5.5, a neutral hydrophilic sieving polymer, and between about 0.5 and about 30 g/L cationic surfactant.

This is a continuation-in-part application, a continuation of application Ser. No. 12/359, 345, filed Jan. 26, 2009.

### REFERENCES CITED

### U.S. Patent Documents:

1) U.S. Pat. No. 4,481,094 Stabilized polyacrylamide gels and system for SDS electrophoresis
2) U.S. Pat. No. 5,089,111 Electrophoretic sieving in gel-free media with dissolved polymers
3) U.S. Pat. No. 5,143,753 Suppression of electroosmosis with hydrolytically stable coatings
4) U.S. Pat. No. 5,213,669 Capillary column containing a dynamically cross-linked composition and method of use
5) U.S. Pat. No. 5,275,708 Cetyltrimethylammonium bromide gel electrophoresis
6) U.S. Pat. No. 5,370,777 Capillary column containing removable separation gel composition and method of use
7) U.S. Pat. No. 5,470,916 Formulations for polyacrylamide matrices in electrokinetic and chromatographic methodologies
8) U.S. Pat. No. 5,552,028 Polymers for separation of biomolecules by capillary electrophoresis
9) U.S. Pat. No. 5,567,292 Polymers for separation of biomolecules by capillary electrophoresis
10) U.S. Pat. No. 5,916,426 Polymers for separation of biomolecules by capillary electrophoresis
11) U.S. Pat. No. 6,355,709 Polymers for separation of biomolecules by capillary electrophoresis
12) 13) U.S. Pat. No. 6,646,084 Polymers for separation of biomolecules by capillary electrophoresis
13) U.S. Pat. No. 7,045,048 Polymers for separation of biomolecules by capillary electrophoresis
14) 20050161329 Multiplexed capillary electrophoresis systems
15) 20020049184 Solution of galactomannans as a sieving matrix in capillary electrophoresis
16) 20040050702 Methods and compositions for capillary electrophoresis (CE)
17) Ser. No. 13/342,031 Separation medium for capillary electrophoresis suppressing electroosmotic flow in bare capillaries and channels.

### OTHER REFERENCES

1. Shapiro, A. L., Vinuela, E., Maizel, J. V. Molecular weight estimation of polypeptide chains by electrophoresis in SDS-polyacrylamide gels. Biochem. Biophys. Res. Commun. 28 (1967) 815-820.
2. Akins, R. E., Levin, P. M., Tuan, R. S. Cetyltrimethylammonium bromide discontinuous gel electrophoresis: M(r)-based separation of proteins with retention of enzymatic activity. Anal. Biochem. 202 (1992) 172-178.
3. Akins, R. E., Tuan, R. S. Separation of proteins using cetyltrimethylammonium bromide discontinuous gel electrophoresis. Mol. Biotech. 1 (1994) 211-228.
4. Akin, D. T., Shapira, R., Kinkade Jr., J. M. The determination of molecular weights of biologically active proteins by cetyltrimethylammonium bromide-polyacrylamide gel electrophoresis. Anal. Biochem. 145 (1985) 170-176.
5. Eley, M. H., Burns, P. C., Kannapell, C. C., Campbell, P. S. Cetyltrimethylammonium bromide polyacrylamide gel electrophoresis: estimation of protein subunit molecular weights using cationic detergents. Anal. Biochem. 92 (1979) 411-419.
6. Panyim, S., Thitipongpanich, R., Supatimusro, D. A simplified gel electrophoretic system and its validity for molecular weight determinations of protein cetyltrimethylammonium complexes. Anal. Biochem. 81 (1977) 320-327.
7. Schick, M. Influence of a cationic detergent on electrophoresis in polyacrylamide gel. Anal. Biochem. 63 (1975) 345-349.
8. Noolandi J., Theory of DNA gel electrophoresis. Adv. Electrophor. 5 (1992) 1-57.
9. Ganzler, K., Greve, K. S., Cohen, A. S., Karger, B. L., Guttman, A., Cooke, N. C. High-Performance Capillary Electrophoresis of SDS Protein Complexes Using UV-Transparent Polymer Networks. Anal. Chem. 64 (1992) 2665-2671.
10. Karim, M. R., Janson, J. C., Takagi, T. Size-dependent separation of proteins in the presence of sodium dodecyl sulfate and dextran in capillary electrophoresis: Effect of molecular weight of dextran. Electrophoresis 15 (1994) 1531-1534.
11. Bae, Y. C., Soane, D. Polymeric Separation Media for Electrophoresis—Cross-Linked Systems or Entangled Solutions. J. Chromatogr. A 652 (1993) 17-22.
12. Dolnik, V., Gurske, W. A., Padua, A. Sieving matrices in capillary electrophoresis: Inflection slope and double reciprocal plot. Electrophoresis 22 (2001) 692-698.
13. Salas-Solano, O., RuizMartinez, M. C., Carrilho, E., Kotler, L., Karger, B. L. A sample purification method for rugged and high performance DNA sequencing by capillary electrophoresis using replaceable polymer solutions. B. Quantitative determination of the role of sample matrix components on sequencing analysis. Anal. Chem. 70 (1998) 1528-1535.
14. Dolnik, V., Gurske, W. A., Padua, A. Galactomannans as a sieving matrix in capillary electrophoresis. Electrophoresis 22 (2001) 707-719.
15. Goetzinger, W., Kotler, L., Carrilho, E., RuizMartinez, M. C., SalasSolano, O., Karger, B. L. Characterization of high molecular mass linear polyacrylamide powder prepared by emulsion polymerization as a replaceable polymer matrix for DNA sequencing by capillary electrophoresis. Electrophoresis 19 (1998) 242-248.
16. Hjerten, S. High-performance electrophoresis: the electrophoretic counterpart of high-performance liquid chromatography. J. Chromatogr. 270 (1983) 1-6.
17. Cohen, A. S., Karger, B. L. High-performance sodium dodecyl sulfate polyacrylamide gel capillary electrophoresis of peptides and proteins. J. Chromatogr. 397 (1987) 409-417.
18. Hjerten, S., Valtcheva, L., Elenbring, K., Eaker, D. High-performance electrophoresis of acidic and basic low-molecular-weight compounds and of proteins in the presence of polymers and neutral surfactants. J. Liq. Chromatogr. 12 (1989) 2471-2499.
19. Sudor, J., Foret, F., Bocek, P. Pressure Refilled Polyacrylamide Columns for the Separation of Oligonucleotides by Capillary Electrophoresis. Electrophoresis 12 (1991) 1056-1058.
20. Heiger, D. N., Cohen, A. S., Karger, B. L. Separation of DNA Restriction Fragments by High-Performance Capillary Electrophoresis with Low and Zero Cross-Linked Polyacrylamide Using Continuous and Pulsed Electric-Fields. J. Chromatogr. 516 (1990) 33-48.
21. Guttman, A., Nolan, J. A., Cooke, N. Capillary Sodium Dodecyl Sulfate Gel Electrophoresis of Proteins. J. Chromatogr. 632 (1993) 171-175.
22. Ganzler, K., Greve, K. S., Cohen, A. S., Karger, B. L., Guttman, A., Cooke, N. C. High-Performance Capillary Electrophoresis of SDS Protein Complexes Using UV-Transparent Polymer Networks. Anal. Chem. 64 (1992) 2665-2671.
23. Izumi, T., Yamaguchi, M., Yoneda, K., Isobe, T., Okuyama, T., Shinoda, T. Use of Glucomannan for the Separation of DNA Fragments by Capillary Electrophoresis. J. Chromatogr. A 652 (1993) 41-46.
24. Kleemiss, M. H., Gilges, M., Schomburg, G. Capillary Electrophoresis of DNA Restriction Fragments with Solutions of Entangled Polymers. Electrophoresis 14 (1993) 515-522.
25. Madabhushi, R. S. Separation of 4-Color DNA-Sequencing Extension Products in Noncovalently Coated Capillaries Using Low-Viscosity Polymer-Solutions. Electrophoresis 19 (1998) 224-230.
26. Lindberg, P., Righetti, P. G., Gelfi, C., Roeraade, J. Electrophoresis of DNA-Sequencing Fragments at Elevated-Temperature in Capillaries Filled with Poly(N-Acryloylaminopropanol) Gels. Electrophoresis 18 (1997) 2909-2914.
27. Chiari, M., Nesi, M., Righetti, P. G. Capillary Zone Electrophoresis of DNA Fragments in a Novel Polymer Network—Poly(N-Acryloylaminoethoxyethanol). Electrophoresis 15 (1994) 616-622.
28. Chiari, M., Riva, S., Gelain, A., Vitale, A., Turati, E. Separations of DNA Fragments by Capillary Electrophoresis in N-Substituted Polyacrylamides. J. Chromatogr. A 781 (1997) 347-355.
29. Motsch, S. R., Kleemiss, M. H., Schomburg, G. Production and Application of Capillaries Filled with Agarose-Gel for Electrophoresis. J. High Resol. Chromatogr. 14 (1991) 629-632.
30. Shapiro, A. L., Maizel, J. V. Molecular weight estimation of polypeptides by SDS-polyacrylamide gel electrophoresis: further data concerning resolving power and general considerations. Anal. Biochem. 29 (1969) 505-514.
31. Weber, K., Osborn, M. The Reliability of Molecular Weight Determinations by Dodecyl Sulfate-Polyacrylamide Gel Electrophoresis. J. Biol. Chem. 242 (1969) 4406-4412.
32. Dunker, A. K., Rueckert, R. R. Observations on molecular weight determinations on polyacrylamide gel. J. Biol. Chem. 244 (1969) 5074-5080.
33. Kresge, N., Simoni, R. D., Hill, R. L. SDS-PAGE to Determine the Molecular Weight of Proteins: the Work of Klaus Weber and Mary Osborn. J. Biol. Chem. 281 (2006) e19-e21.
34. Reynolds, J. A., Tanford, C. Binding of Dodecyl Sulfate to Proteins at High Binding Ratios. Possible Implications for the State of Proteins in Biological Membranes. Proc. Natl. Acad. Sci. U.S.A. 66 (1970) 1002-1007.
35. Williams, J. G., Gratzer, W. B. Limitations of the detergent polyacrylamide gel electrophoresis method for molecular weight determination of proteins. J. Chromatogr. 57 (1971) 121-125.
36. Nelson, C. A. The binding of detergents to proteins. J. Biol. Chem. 246 (1971) 3895-3901.
37. Dolnik, V., Cobb, K. A., Novotny, M.: Preparation of polyacrylamide gel-filled capillaries for capillary electrophoresis. J. Microcol. Sep. 3 (1991) 155-159.
38. Guttman, A., Horvath, J., Cooke, N. Influence of Temperature on the Sieving Effect of Different Polymer Matrices in Capillary SDS Gel Electrophoresis of Proteins. Anal. Chem. 65 (1993) 199-203.
39. Werner, W. E., Demorest, D. M., Stevens, J., Wiktorowicz, J. E. Size-Dependent Separation of Proteins Denatured in SDS by Capillary Electrophoresis Using a Replaceable Sieving Matrix. Anal. Biochem. 212 (1993) 253-258.
40. Guttman, A., Shieh, P., Lindahl, J., Cooke, N. Capillary sodium dodecyl sulfate gel electrophoresis of proteins 0.2. On the Ferguson method in polyethylene oxide gels. J. Chromatogr. A 676 (1994) 227-231.
41. Tsuji, K. Sodium dodecyl sulfate polyacrylamide gel- and replaceable polymer-filled capillary electrophoresis for molecular mass determination of proteins of pharmaceutical interest. J. Chromatogr. B 662 (1994) 291-299.
42. Guttman, A. On the separation mechanism of capillary sodium dodecyl sulfate-gel electrophoresis of proteins. Electrophoresis 16 (1995) 611-616.
43. Nakatani, M., Shibukawa, A., Nakagawa, T. Effect of temperature and viscosity of sieving medium on electrophoretic behavior of sodium dodecyl sulfate-proteins on capillary electrophoresis in presence of pullulan. Electrophoresis 17 (1996) 1210-1213.
44. Nakatani, M., Shibukawa, A., Nakagawa, T. High-Performance Capillary Electrophoresis of SDS-Proteins Using Pullulan Solution as Separation Matrix. J. Chromatogr. A 672 (1994) 213-218.
45. Hu, S., Ye, Y. L., Surh, G., Clark, J. I., Dovichi, N. J. Analysis of Proteins by Capillary Sds-Dalt Electrophoresis With Laser-Induced Fluorescence Detection. LC GC (Europe) 15 (2002) 166-+.
46. Craig, D. B., Polakowski, P. M., Arriaga, E., Wong, J. C. Y., Ahmadzadeh, H., Stathakis, C., Dovichi, N. J. Sodium dodecyl sulfate-capillary electrophoresis of proteins in a sieving matrix utilizing two-spectral channel laser-induced fluorescence detection. Electrophoresis 19 (1998) 2175-2178.
47. Hunt, G., Nashabeh, W. Capillary electrophoresis sodium dodecyl sulfate nongel sieving analysis of a therapeutic recombinant monoclonal antibody: A biotechnology perspective. Anal. Chem. 71 (1999) 2390-2397.
48. Salas-Solano, O., Tomlinson, B., Du., S., Parker, M., Strahan, A., Ma, S. Optimization and validation of a quantitative capillary electrophoresis sodium dodecyl sulfate method for quality control and stability monitoring of monoclonal antibodies. Anal. Chem. 78 (2006) 6583-6594.
49. Yao, S., Anex, D. S., Caldwell, W. B., Arnold, D. W., Smith, K. B., Schultz, P. G. SDS capillary gel electrophoresis of proteins in microfabricated channels. Proc. Natl. Acad. Sci. U.S.A. 96 (1999) 5372-5377.
50. Bousse, L., Mouradian, S., Minalla, A., Yee, H., Williams, K., Dubrow, R. Protein sizing on a microchip. Anal. Chem. 73 (2001) 1207-1212.
51. Werner, W. E., Demorest, D. M., Wiktorowicz, J. E. Automated Ferguson analysis of glycoproteins by capillary electrophoresis using a replaceable sieving matrix. Electrophoresis 14 (1993) 759-763.
52. Guttman, A., Nolan, J. A., Cooke, N. Capillary Sodium Dodecyl Sulfate Gel Electrophoresis of Proteins. J. Chromatogr. 632 (1993) 171-175.
53. Guttman, A. Capillary sodium dodecyl sulfate-gel electrophoresis of proteins. Electrophoresis 17 (1996) 1333-1341.
54. Guttman, A., Nolan, J. Comparison of the Separation of Proteins by Sodium Dodecyl Sulfate-Slab Gel Electrophoresis and Capillary Sodium Dodecyl Sulfate-Gel Electrophoresis. Anal. Biochem., 221 (1994) 285-289.
55. Nozaki, Y., Reynolds, J. A., Tanford, C. The Interaction of a Cationic Detergent with Bovine Serum Albumin and Other Proteins. J. Biol. Chem., 249 (1974) 4452-4459.
56. Reijenga, J. C., Aben, G. V. A., Verheggen, T. H. A. M., Everaerts, F. M. Effect of electroosmosis on detection in isotachophoresis. J. Chromatogr., 260 (1983) 241-254.
57. Tsuda, T. Modification of electroosmotic flow with cetyltrimethylammonium bromide in capillary zone electrophoresis. HRC CC, J. High Res. Chromatogr. Chromatogr. Commun., 10 (1987) 622-624.
58. Corradini, D. Buffer additives other than the surfactant sodium dodecyl sulfate for protein separations by capillary electrophoresis. J. Chromatogr. B, 699 (1997) 221-256.
59. Ding, W. L., Fritz, J. S. Separation of Basic-Proteins and Peptides by Capillary Electrophoresis Using a Cationic Surfactant. J. High Resol. Chromatogr., 20 (1997) 575-580.
60. Chiari, M., Damin, F., Reijenga, J. C. Characterization of poly(dimethylacrylamide) and the combination of poly(vinyl alcohol) and cetyltrimethylanlmonium bromide as dynamic electroosmotic flow suppression agents in capillary electrophoresis. J. Chromatogr. A, 817 (1998) 15-23.
61. Ong, C. P., Ng, C. L., Lee, H. K., Li, S. F. Y. The Use of Mixed Surfactants in Micellar Electrokinetic Chromatography. Electrophoresis, 15 (1994) 1273-1275.
62. Gilbert, H. F. Thiol/disulfide exchange equilibria and disulfide bond stability. Meth. Enzymol., 251 (1995) 8-28.
63. Menger, F. M., Keiper, J. S. Gemini surfactants. Angew. Chem., 39 (2000) 1906-1920.
64. Ruegg, U. T., Rudinger, J., Reductive cleavage of cystine disulfides with tributylphosphine. Meth. Enzymol. 47 (1977) 111-126.

### FIELD OF THE INVENTION

The present invention relates to size separation of proteins by capillary electrophoresis in sieving media, wherein one or more cationic surfactants form charged complexes with the proteins and equalize their surface charge density, making their migration in sieving media independent of their intrinsic charge and thus allowing their size separation and molecular-weight determination. Specifically, the invention is directed to capillary sieving electrophoresis of proteins in the presence of cationic surfactants at low pH.

### BACKGROUND OF THE INVENTION

### Electrophoresis in Sieving Media

Electrophoretic sieving media are used to size separate biopolymers: nucleic acids, polysaccharides, and proteins. They provide a system of obstacles in the electrophoretic migration path so that migrating biopolymers collide with the obstacles and these collisions retard their apparent migration velocity. Larger molecules and particles are retarded in their migration more than small molecules. The first electrophoretic sieving media were starch and polyacrylamide gel. Nucleic acids are equally ionized at non-acidic pH and need not be modified to size separate during electrophoretic migration in sieving media. On the other hand, protein ionization and charge significantly vary depending on the amino acid composition. Therefore, native proteins are not size separated in sieving media in absence of ionic surfactants. However, when heated with an ionic surfactant, proteins denature and bind the surfactants, generating complexes with more or less equal surface charge density. These complexes migrate in sieving media according to their size. Ionic surfactants such as sodium dodecyl sulfate (SDS) (Shapiro, Vinuela, and Maizel, 1967), cetyltrimethylammonium bromide (CTAB) (Panyim, Thitipongpanich, and Supatimusro, 1977), cetylpyridinium chloride (Schick, 1975) have been used to equalize the surface charge density of proteins prior electrophoresis.

### Slab Gel Electrophoresis

SDS electrophoresis in polyacrylamide slab gel (SDS PAGE) was the first method separating proteins according to their size (Shapiro, Vinuela, and Maizel, 1967^{;} Shapiro and Maizel, 1969^{;} Weber and Osborn, 1969^{;} Dunker and Rueckert, 1969). Formation of SDS-protein complexes is independent of ionic strength (Reynolds and Tanford, 1970). However, some proteins exhibit an anomalous migration in SDS PAGE (Shapiro, Vinuela, and Maizel, 1967^{;} Williams and Gratzer, 1971). The anomalous migration of acidic proteins in SDS PAGE was, however, normalized by esterification of carboxyl groups (Williams and Gratzer, 1971) suggesting insufficient surfactant binding. This hypothesis was corroborated by an observation that some acidic proteins, such as pepsin, papain, and glucose oxidase do not bind measurable amount of SDS (Nelson, 1971).

Shortly after the invention of SDS PAGE, a method separating proteins by polyacrylamide gel electrophoresis (PAGE) in the presence of cationic surfactants was described (Williams and Gratzer, 1971). A study based on observations of behavior of protein-cationic-surfactant-complexes followed, predicting a failure of the electrophoresis in the presence of cationic surfactants to determine molecular weights of proteins (Nozaki, Reynolds, and Tanford, 1974). Later, cetylpyridinium chloride (Schick, 1975) and cetyltrimethylammonium bromide (Akins, Levin, and Tuan, 1992^{;} Akins and Tuan, 1994^{;} Akin, Shapira, and Kinkade Jr., 1985^{;} Eley, Burns, Kannapell, and Campbell, 1979^{;} Panyim, Thitipongpanich, and Supatimusro, 1977) were used for size separations of proteins by PAGE. Several protocols have been developed to denature proteins with cetyltrimethylammonium bromide (Akins, Levin, and Tuan, 1992^{;} Akins and Tuan, 1994^{;} Akin, Shapira, and Kinkade Jr., 1985^{;} Eley, Burns, Kannapell, and Campbell, 1979^{;} Panyim, Thitipongpanich, and Supatimusro, 1977), including a protocol without any heating of the sample (Akins, Levin, and Tuan, 1992).

### Capillary Electrophoresis

When electrophoresis of proteins in sieving media was transferred from slab gels into capillaries, crosslinked polyacrylamide gel was initially used as the sieving matrix (Hjerten, 1983^{;} Cohen and Karger, 1987). When linear hydrophilic polymers were introduced as a replaceable sieving matrix for separation of polynucleotides (Hjerten, Valtcheva, Elenbring, and Eaker, 1989), various polymers were introduced for electrophoretic size separation of biopolymers, including linear polyacrylamide (Ganzler, Greve, Cohen, Karger, Guttman, and Cooke, 1992a^{;} Sudor, Foret, and Bocek, 1991^{;} Heiger, Cohen, and Karger, 1990), poly(ethylene oxide) (Guttman, Nolan, and Cooke, 1993), dextran (Ganzler, Greve, Cohen, Karger, Guttman, and Cooke, 1992b), guaran (Dolnik, Gurske, and Padua, 2001a), glucomannan (Izumi, Yamaguchi, Yoneda, Isobe, Okuyama, and Shinoda, 1993), poly(vinyl alcohol) (Kleemiss, Gilges, and Schomburg, 1993), poly(dimethyl acrylamide) (Madabhushi, 1998), poly(hydroxypropyl acrylamide) (Lindberg, Righetti, Gelfi, and Roeraade, 1997), poly(ethoxyethyl acrylamide) (Chiari, Nesi, and Righetti, 1994^{;} Chiari, Riva, Gelain, Vitale, and Turati, 1997), agarose (Motsch, Kleemiss, and Schomburg, 1991), and pullulan (Nakatani, Shibukawa, and Nakagawa, 1994^{;} Nakatani, Shibukawa, and Nakagawa, 1996). Capillary size separations of proteins were performed exclusively by SDS capillary sieving electrophoresis (CSE). The method was also modified for size separation of proteins on microchip (Yao, Anex, Caldwell, Arnold, Smith, and Schultz, 1999) with poly(dimethyl acrylamide) as a sieving polymer (Yao, Anex, Caldwell, Arnold, Smith, and Schultz, 1999). (Bousse, Mouradian, Minalla, Yee, Williams, and Dubrow, 2001) Capillary electrophoresis brought a number of advantages as compared to electrophoresis in slab gel: faster analysis, automation, higher separation efficiency, and higher detection sensitivity. Nevertheless, a small size of capillaries emphasized the effect of the capillary wall: typically the used fused silica capillaries contained ionized silanol groups on their internal surface, resulting in strong wall adsorption, significant electroosmotic flow, eddy migration, and consequent mediocre separation efficiency. Electroosmotic flow was eventually suppressed by applying a hydrolytically stable neutral coating on the capillary wall (U.S. Pat. No. 5,143,753). Nevertheless, in SDS CSE, SDS may adsorb on the neutral coating and generate secondary electroosmotic flow resulting in mediocre reproducibility and separation efficiency.

In SDS CSE, electroosmotic flow was also suppressed by applying high-concentration Tris-borate buffer (U.S. Patent application 20040050702). Borate complexes with polyol bases were also used to suppress electroosmotic flow in other CE applications (U.S. patent application Ser. No. 13/342,031).

Hypothetically, electroosmotic flow in SDS CSE could be also suppressed by reducing pH of the sieving medium and a consequent suppression of the silanol ionization in the capillary wall. However, SDS binding of proteins is weaker at pH<6 and SDS electrophoresis at this pH results in significantly broader peaks (Gilbert, 1995) excluding this alternative from a real world practice.

### Polymer Solutions as a Sieving Matrix

When migration of biopolymers in sieving media was studied in a greater detail, three distinct migration modes were identified, depending on the pore size of the sieving matrix and size of the polyelectrolyte: Ogston mode, reptation without stretching, and reptation with stretching (Noolandi J., 1992). In capillary electrophoresis, where sieving polymers serve as the sieving matrix, the pore size of the sieving matrix is fine tuned by changing the concentration of the sieving polymer (Ganzler, Greve, Cohen, Karger, Guttman, and Cooke, 1992a^{;} Karim, Janson, and Takagi, 1994). Molecular weight of the sieving polymer also plays an important role, particularly in separation of large polyelectrolytes migrating in the reptation-with-stretching mode (Bae and Soane, 1993). High-molecular-weight polymers are typically better sieving matrices than those with low molecular weight (Dolnik, Gurske, and Padua, 2001b) although blends of polymers of high and low molecular weight were also advocated and used (Salas-Solano, RuizMartinez, Carrilho, Kotler, and Karger, 1998). Stiffness and branching of the sieving polymers affects the sieving properties of the sieving matrix as well: branched polysaccharides such as dextran or Ficoll® exhibit rather limited sieving and are suitable more for separation of polyelectrolytes migrating in the Ogston mode. Stiff polysaccharides such as hydroxyethyl cellulose, guaran, and locust bean gum (Dolnik, Gurske, and Padua, 2001a) form a rigid sieving matrix at lower molecular weights and/or concentrations than easily bending synthetic polymers such as linear polyacrylamide. In practical CSE analysis, viscosity is a limiting factor since pressure as high as 900 psi is necessary to replace a viscous sieving matrix in the capillary. The viscosity of the sieving matrix makes a practical limit when increasing the concentration and molecular weight of the sieving polymer. In DNA sequencing by capillary electrophoresis, the weight molecular weight (M_{w}, further molecular weight) of linear polyacrylamide used as a sieving polymer, exceeded 10,000,000 (Goetzinger, Kotler, Carrilho, RuizMartinez, SalasSolano, and Karger, 1998). In CSE of proteins, most proteins were separated in sieving media with large pore size and sieving polymers typically did not exceed molecular weight of 1 million. To provide efficient sieving, the sieving polymers should have molecular weight about 100,000 and more. However, this value is rather arbitrary, as lower-molecular-weight polymers can be prepared at a higher concentration and still keep the viscosity of the solution at an acceptable level. Stiff polysaccharides, such as hydroxyethyl cellulose, guaran, or locust bean gum can be used even at a lower molecular weight. On the other hand, branched polysaccharides, such as dextran, are preferably used at high molecular weight (2 million and higher).

### Cationic Surfactant in Capillary Electrophoresis

Cetyltrimethylammonium bromide was used in capillary electrophoresis for two purposes: a) as a dynamic coating to reverse the direction of electroosmotic flow (Reijenga, Aben, Verheggen, and Everaerts, 1983^{;} Tsuda, 1987^{;} Corradini, 1997^{;} Ding and Fritz, 1997^{;} Chiari, Damin, and Reijenga, 1998), b) as a pseudostationary phase in capillary electrokinetic micellar chromatrography (Ong, Ng, Lee, and Li, 1994). None cationic surfactant has been used for size separation of proteins by capillary sieving electrophoresis yet. An interesting group of potential cationic surfactants are gemini surfactants (Menger and Keiper, 2000) with two amino groups connected by an aliphatic arm but they have not been used in capillary electrophoresis yet.

### SUMMARY OF THE INVENTION

The present invention is suitable for a fast, quantitative, and highly reproducible size separation of proteins by means of capillary sieving electrophoresis. Disclosed herein are a composition of a separation medium and a protein denaturing solution, and a method of capillary sieving electrophoresis in the presence of a cationic surfactant for size separation of proteins with molecular weight in the range between about 14,000 and about 500,000. In the preferred embodiment, the separation medium comprises an acidic buffer that keeps pH in the range between about 3 and about 5, a hydrophilic sieving polymer with a moderate viscosity, and between about 0.5 and about 30 g/L cationic surfactant.

### BRIEF DESCRIPTION OF THE DRAWINGS

FIG. 1 shows separation of a model protein mixture. Separation medium: 100 mM β-alanine, 100 mM glutamic acid, 1 g/L cetyldimethylethylammonium bromide (CDMEAB), 16 g/L poly(ethylene oxide) (M_{w} 400,000). Coated capillary Guarant™ (Alcor BioSeparations LLC, Palo Alto, Calif., U.S.A.): total length=335 mm, effective length=250 mm, ID=75 μm, OD=360 μm. Voltage: +10 kV. Detection: UV absorption at 214 nm. Electrokinetic injection: 6 s at +8 kV. Sample: about 1 g/L proteins in 10 g/L CDMEAB, 100 mM KCl, 10 g/L dithiothreitol (DTT) heated 5 min at 95° C. (lysozyme), 2 min at 95° C. (all other proteins).

FIG. 2 is the plot of protein mobility vs. their logarithmic molecular weight calculated from the electropherogram in FIG. 1.

FIG. 3 shows an electropherogram of model proteins in bare capillary. Separation medium: 100 mM β-alanine, 100 mM 2-hydroxisobutyric acid, 2 g/L cetyltrimethylammonium chloride (CTAC), 50 g/L dextran (M_{w} 2,000,000). Flush solution: 10 g/L poly(ethylene oxide) (M_{w} 7,000,000), 400 mM β-alanine, 400 mM 2-hydroxisobutyric acid. Capillary: bare, total length=335 mm, effective length=250 mm, ID=75 μm, OD=360 μm. Voltage: +10 kV. Electrokinetic injection: 15 s at +8 kV. Sample: 0.8 g/L model proteins in 100 mM KCl. 10 g/L CTAC, 4 g/L tris-(carboxyethyl)phosphine (TCEP), 5 min incubated at 70° C.

FIG. 4 displays the separation of BSA oligomers. Injection: 15 s at +8 kV. Sample: 10 g/L BSA in 1 g/L cetyldimethylethylammonium bromide (CDMEAB). All other experimental conditions were same as in FIG. 1.

FIG. 5 presents the plot of the mobility vs. logarithmic molecular weight for BSA oligomers as calculated from the electropherogram in FIG. 4.

FIG. 6 shows 10 overlaid electropherograms of model proteins from 10 consecutive runs. Separation medium: 100 mM γ-aminobutyric acid, 100 mM glutamic acid, 25 g/L CTAB, 20 g/L poly(ethylene oxide) (M_{w} 200,000). Capillary: bare capillary, total length=335 mm, effective length=250 mm, ID=75 μm, OD=360 μm. Voltage: +10 kV. Electrokinetic injection: 3 s at +3 kV. Sample: 0.8 g/L model proteins in 30 mM CTAB, 60 mM DTT, 5 min incubated at 95° C. (lysozyme), 2 min at 95° C. (all other proteins).

FIG. 7 depicts calibration curves of model proteins with electrokinetic injection 30 s at +10 kV. Sample denaturing solution: 10 g/L CDMEAB, 10 g/L DTT, 100 mM KCl, 5 min incubated at 95° C. (lysozyme), 2 min at 95° C. (all other proteins). Separation medium: 100 mM β-alanine, 100 mM glutamic acid, 1 g/L CDMEAB, 20 g/L poly(ethylene oxide) (M_{w} 200,000). Capillary Guarant™ (Alcor BioSeparations, Palo Alto, Calif., U.S.A.): total length=335 mm, effective length=250 mm, ID=75 μm, OD=360 μm. Voltage: +10 kV. ♦—lysozyme, x—BSA (monomer), ▪—β-lactoglobulin, Δ—ovalbumin.

### DETAILED DESCRIPTION OF THE INVENTION

### Cationic Surfactants

A cationic surfactant used in the sieving matrix should exhibit a sufficient solubility in water and, simultaneously it should strongly bind proteins. Primary, secondary, tertiary or quaternary amines with one or more long aliphatic chains are suitable cationic surfactants for CSE of proteins. Typically longer aliphatic chains are preferred because then the surfactant binds proteins more strongly. However, solubility of the cationic surfactant in water may be a limiting factor for its practical use. Cationic surfactants suitable for capillary sieving electrophoresis of proteins contain one or more of the following cations: octadecyldimethylethylammonium, cetyldimethylethylammonium, tetradecyldimethylethylammonium, dodecyldimethylethylammonium, octadecyltrimethylammonium, cetyltrimethylammonium, tetradecyltrimethylammonium, dodecyltrimethylammonium, octadecylpyridinium, tetradecylpyridinium, dodecylpyridinium, octadecylammonium, cetylammonium, tetradecylammonium, dodecylammonium, decylammonium, didodecyldimethylammonium, and a cationic gemini surfactant alkanediyl-.α.,.ω.-bis(dimethylalkylammonium), with a formula CₘH₂ₘ₊₁(CH₃)₂N⁺(CH₂)ₛN⁺(CH₃)₂CₘH₂ₘ₊₁, m being 12, 14, 16, or 18 and s being 2, 3, 4, 5, 6, 7, or 8.

The counter anion in the cationic surfactant can be an inorganic anion such as chloride, bromide, sulfate, bicarbonate, etc. The nature of the counter anion plays a significant role. In capillary sieving electrophoresis of monoclonal antibodies, a broad system peak appears when cetyltrimethylammonium bromide is used as the cationic surfactant. With cetyltrimethylammonium chloride, the system peak is much smaller and such a sieving medium is more preferred.

### Acid Buffer

Size separations of proteins in capillary format based on SDS frequently suffer from mediocre separation efficiency and lower reproducibility of qualitative and quantitative analysis. Capillary sieving electrophoresis of proteins with cationic surfactant at low pH, where ionization of silanol groups in the capillary wall is reduced can perform better. The separation medium contains a cationic surfactant, a sieving polymer, and an acidic buffer with pH below 5.

However, below pH 3, the high-mobility H⁺ ion contributes significantly to the conductivity of the sieving matrix. This results in elevated Joule heat and overheating of the capillary. Experiments show, keeping the pH of the sieving matrix at about pH 4 is the best solution.

One possibility is to use a free weak acid, e.g., acetic acid, as the only electrolyte. Another option is to use fully ionized cation, e.g., Tris, with a buffering anion, e.g., formate, acetate, propionate, butyrate, capronate, valproate, pimelate, fumarate, maleate, succinate, glutarate, adipate, malate, tartrate, glycolate, lactate, 2-hydroxybutyrate, 2-hydroxyisobutyrate, citrate, nicotinate, glutamate, and aspartate. pH can be also kept at a adequate value with a buffering cation, e.g., β-alanine, γ-aminobutyric acid (GABA), glycine, ε-aminocaproic acid, or nicotinamide and a fully ionize anion. Probably the most attractive buffering option is an equimolar mixture of a weak base and a weak acid, having close pK's, e.g., GABA (pK 4.0) and glutamic acid (pK 4.2), β-alanine (pK 3.6) and glutamic acid (pK 4.2), and β-alanine (pK 3.6) and 2-hydroxy-isobutyric acid (pK 3.9). If dicarboxylic or tricarboxylic acids are used as a buffer, they have to be used at pH, where only one carboxylic group is partly dissociated. Other factors than pK, which may be difficult to predict from the physicochemical properties of the buffers, may be also important: Buffers containing β-alanine show better separation efficiency than buffers with GABA, but in its presence, proteins more strongly adsorb on the wall. This results in a minor but discernable baseline elevation when proteins migrate through the detection cell.

### Sieving Polymer

The sieving matrix enables size separation of proteins. It provides obstacles in the migration path and forces proteins-surfactants complexes to electrophoretically migrate according to their size. The sieving polymer should (i) be soluble in water, (ii) be non-ionic, (iii) not significantly bind cationic surfactants, (iv) should have sufficient molecular weight and provide sufficient sieving, (v) not absorb UV light if UV detection is used. The sieving properties of the matrix can be fine tuned by changing the molecular weight and concentration of the sieving polymer. Higher concentration and/or higher molecular weight of the sieving polymer results in smaller pores, i.e., in more efficient sieving but also in longer migration times. Simultaneously, the viscosity rises and may prevent fast replacement of the sieving matrix in the capillary. Linear polysaccharides, such as hydroxyethyl cellulose, hydroxypropyl cellulose, guaran, locust bean gum, glucomannan, and pullulan, have a stiff molecule and will be typically used at concentrations from about 4 to about 60 g/L and molecular weight from about 20,000 to about 500,000. Hydrophilic synthetic polymers, such as linear polyacrylamide, poly(dimethyl acrylamide), poly(hydroxyethyl acrylamide), poly(hydroxypropyl acrylamide), poly(ethoxyethyl acrylamide), poly(vinyl alcohol), poly(vinyl pyrrolidone), and poly(ethylene oxide) will be effective sieving polymers at concentration from about 8 to about 80 g/L and molecular weight from about 100,000 to about 1 million. Branched polysaccharides such as dextran or Ficoll® are less efficient sieving polymers and have to be used at concentration from about 100 to about 400 g/L, at molecular weight of about 2 million. UV absorbing polymers, such as poly(vinyl pyrrolidone) will not properly work in CSE with UV detection but may be used in CSE with laser induced fluorescence detection.

### What is Disclosed:

We disclose here a protein denaturing solution for sample preparation prior capillary electrophoretic size separation of proteins, comprising:
from about 1 g/L to about 30 g/L cationic surfactant selected from the group consisting of cetyldimethylethylammonium bromide, cetyltrimethylammonium chloride, cetyltrimethylammonium bromide, tetradecyltrimethylammonium bromide, dodecyltrimethylammonium bromide, cetylamine, tetradecylamine, dodecylamine, decylamine, and didodecyldimethylammonium bromide;
a reducing agent selected from the group consisting of from about 1 g/L to about 30 g/L dithiothreitol, from about 20 mM to about 50 mM tris-(carboxyethyl)phosphine, and from about 1 g/L to about 30 g/L 2-mercaptoethanol; and
from about 20 mM to about 300 mM electrolyte selected from the group consisting of potassium chloride, potassium phosphate, and ammonium acetate.

Further we disclose here a protein denaturing solution for sample preparation prior capillary electrophoretic size separation of proteins of claim 1, wherein said protein denaturing solution comprises about 10 g/L cetyldimethylethylammonium bromide, about 100 mM potassium phosphate, and about 10 g/L dithiothreitol.

We disclose also a protein denaturing solution for sample preparation of proteins prior capillary electrophoretic size separation, wherein said protein denaturing solution comprises about 10 g/L cetyldimethylethylammonium bromide, about 100 mM potassium chloride, and about 10 g/L dithiothreitol.

Further we disclose here a protein denaturing solution for sample preparation of proteins prior capillary electrophoretic size separation, wherein said protein denaturing solution comprises about 10 g/L cetyltrimethylammonium chloride, about 100 mM potassium phosphate, and about 10 g/L dithiothreitol.

We also disclose here a protein denaturing solution for sample preparation of proteins prior capillary electrophoretic size separation, wherein said protein denaturing solution comprises about 10 g/L cetyltrimethylammonium chloride, about 100 mM potassium phosphate, and from about 10 mM to about 50 mM tris-(carboxyethyl)phosphine.

Further we disclose a protein denaturing solution for sample preparation of proteins prior capillary electrophoretic size separation, wherein said protein denaturing solution comprises about 10 g/L cetyltrimethylammonium bromide, about 100 mM potassium chloride, and from about 20 mM to about 50 mM tris-(carboxyethyl)phosphine.

We disclose here a method of sample preparation prior capillary electrophoretic size separation of proteins, wherein a protein sample is dissolved in the protein denaturation solution and incubated at temperature from about 60 to about 100° C. for a time interval from about 1 min to about 20 min.

Further we disclose here a method of sample preparation prior capillary electrophoretic size separation of proteins, wherein a protein sample is dissolved in the protein denaturation solution and incubated at about 90° C. for about 2 min.

We also disclose here a method of sample preparation prior capillary electrophoretic size separation of proteins, wherein a protein sample is dissolved in the protein denaturation solution and incubated at about 70° C. for about 10 min.

Further we disclose here a separation medium for capillary electrophoretic size separation of proteins, consisting essentially of:
a cationic surfactant, selected from the group of surfactants consisting of: cetyldimethylethylammonium bromide, tetradecyltrimethylammonium bromide, dodecyltrimethylammonium bromide, cetylamine, tetradecylamine, dodecylamine, decylamine, and didodecyldimethylammonium bromide;
   an acid buffer having pH in the range from about 3 to about 5.5, wherein said acid buffer comprises at least one of the following buffering substances selected from the group consisting of:
glycine, β-alanine, γ-aminobutyric acid, δ-aminovaleric acid, ε-aminocaproic acid, nicotinamide, formate, acetate, propionate, lactate, 2-hydroxybutyrate, 2-hydroxyisobutyrate, nicotinate, glutamate, and aspartate; and
   a sieving polymer, wherein said sieving polymer is selected from the group consisting of:
linear polyacrylamide, poly(dimethyl acrylamide), poly(hydroxyethyl acrylamide), poly(hydroxypropyl acrylamide), poly(vinyl alcohol), poly(vinyl pyrrolidone), hydroxyethyl cellulose, guaran, locust bean gum, glucomannan, pullulan, and dextran.

We disclose here a separation medium for capillary electrophoretic size separation of proteins, wherein said cationic surfactant is cetyldimethylethylammonium bromide in the concentration range from about 0.5 g/L to about 30 g/L.

Further we disclose here a separation medium for capillary electrophoretic size separation of proteins, wherein said cationic surfactant is tetradecyltrimethylammonium bromide in the concentration range from about 0.5 g/L to about 30 g/L.

We disclose here a separation medium for capillary electrophoretic size separation of proteins, wherein said acid buffer comprises from about 20 mM to about 200 mM β-alanine, and from about 20 mM to about 200 mM glutamic acid.

Further we disclose here a separation medium for capillary electrophoretic size separation of proteins, wherein said acid buffer comprises from about 20 mM to about 200 mM γ-aminobutyric acid, and from about 20 mM to about 200 mM glutamic acid.

We also disclose here a separation medium for capillary electrophoretic size separation of proteins, wherein said acid buffer comprises from about 20 mM to about 200 mM β-alanine and from about 20 mM to about 200 mM 2-hydroxyisobutyric acid.

Further we disclose here a separation medium for capillary electrophoretic size separation of proteins, consisting essentially of:
from about 8 g/L to about 30 g/L polyacrylamide, having molecular weight from about 100,000 to about 1,000,000;
from about 20 mM to about 200 mM γ-aminobutyric acid;
from about 20 mM to about 200 mM glutamic acid; and
from about 0.5 g/L to about 30 g/L cetyldimethylethylammonium bromide.

We disclose here a separation medium for capillary electrophoretic size separation of proteins, consisting essentially of 15 g/L polyacrylamide having molecular weight 600,000-1 million, 100 mM β-alanine, 100 mM glutamic acid, and 1 g/L cetyldimethylethylammonium bromide.

Further we disclose here a separation medium for capillary electrophoretic size separation of proteins, consisting essentially of:
from about 60 g/L to about 120 g/L dextran, having molecular weight about 2,000,000;
from about 20 mM to about 200 mM γ-aminobutyric acid;
from about 20 mM to about 200 mM glutamic acid; and
from about 0.5 g/L to about 30 g/L cetyldimethylethylammonium bromide.

We disclose here a separation medium for capillary electrophoretic size separation of proteins, consisting essentially of 100 g/L dextran having molecular weight 2,000,000, 100 mM γ-aminobutyric acid, 100 mM glutamic acid, and 2 g/L cetyldimethylethylammonium chloride.

Further we disclose here a method for capillary electrophoretic size separation of proteins, comprising steps:
sample preparation, wherein a protein sample is dissolved in said protein denaturation solution and incubated at temperature from about 60 to about 100° C. with duration from about 1 min to about 20 min;
flushing the separation capillary with a solution comprising from about 5 g/L to about 30 g/L polyethylene oxide to form a dynamic coating;
filling the separation capillary with said separation medium for capillary electrophoretic size separation of proteins;
sample injection, with the injection voltage from about 0.5 kV to about 12 kV applied for from about 1 to about 60 s;
separation, with the separation voltage from about 1 kV to about 20 kV applied for about 5 minute to about 30 minutes;
detection, wherein the absorption of monochromatic light having wavelength from about 210 nm to about 220 nm is measured and plotted in an electropherogram.

We also disclose here a method for capillary electrophoretic size separation of proteins, wherein said sieving polymer in said separation medium for capillary electrophoretic size separation of proteins is about 100 g/L dextran with molecular weight of about 2 million.

Further we disclose here a method for capillary electrophoretic size separation of proteins, wherein said sieving polymer in said separation medium for capillary electrophoretic size separation of proteins is from about 8 g/L to about 30 g/L polyacrylamide, having molecular weight from about 100,000 to about 1,000,000.

### EXAMPLES

The separations described in these examples were performed in 3D CE capillary electrophoresis instrument at 20° C. in a bare or coated capillary of internal diameter 75 μm and outer diameter 360 vm with UV detection at 214 nm.

### Example 1

### Preparation and Composition of the Sieving Matrix

The separation medium for capillary sieving electrophoresis with a cationic surfactant was formulated to contain cetyldimethylethylammonium bromide (CDMEAB) or cetyltrimethylammonium chloride (CTAC) as the cationic surfactant, polyacrylamide, dextran, or poly(ethylene oxide) (PEO) as the sieving matrix, β-alanine or γ-aminobutyric acid as the buffering co-ion, and 2-hydroxyisobutyric acid or glutamic acid as the buffering counter-ion. Formulation with β-alanine were designed for high resolution separations, formulations with γ-aminobutyric acid were preferred where straight baseline was necessary. The specific formulations contained:
a) 2 g/L CTAC, 100 mM γ-aminobutyric acid, 100 mM glutamic acid, pH about 4.2, and 15 g/L polyacrylamide (M_{w} 600,000-1,000,000);
b) 2 g/L CTAC, 100 mM γ-aminobutyric acid, 100 mM glutamic acid, pH about 4.2, and 15 g/L polyacrylamide (M_{w} 600,000-1,000,000);
c) 2 g/L CDMEAB, 100 mM β-alanine, 100 mM 2-hydroxybutyric acid, pH about 3.7, and 20 g/L PEO (M_{w} 200,000);
d) 2 g/L CDMEAB, 100 mM γ-aminobutyric acid, 100 mM glutamic acid, pH about 4.2, and 20 g/L PEO (M_{w} 200,000);
e) 2 g/L CDMEAB, 100 mM β-alanine, 100 mM 2-hydroxyisobutyric acid, pH about 3.7, and 20 g/L PEO (M_{w} 200,000); x
f) 2 g/L CDMEAB, 100 mM β-alanine, 100 mM 2-hydroxyisobutyric acid, pH about 3.7, and 100 g/L dextran (M_{w} 2,000,000);
g) 2 g/L CDMEAB, 100 mM γ-aminobutyric acid, 100 mM glutamic acid, pH about 4.2, and 100 g/L dextran (M_{w} 2,000,000);
h) 2 g/L CTAC, 100 mM β-alanine, 100 mM glutamic acid, pH about 3.7, and 15 g/L polyacrylamide (M_{w} 600,000-1,000,000);

### Example 2

### Composition of Sample Denaturing Solution and Method of Sample Preparation

Several compositions of the sample denaturing solution were formulated to enable protein quantitation with electrokinetic injection:
a) 10 g/L CDMEAB, 100 mM KCl, and 10 g/L dithiotreitol (DTT);
b) 10 g/L CDMEAB, 100 mM potassium phosphate, and 10 g/L DTT;
c) 10 g/L CTAC, 100 mM potassium phosphate, and 10 g/L DTT;
d) 10 g/L CTAC, 100 mM potassium phosphate, and 10 g/L DTT,
e) 10 g/L CTAB, 100 mM KCl, and 10 g/L DTT;
f) 10 g/L CTAB, 100 mM potassium phosphate, and 10 g/L DTT;
g) 10 g/L CDMEAB, 100 mM KCl, and 50 mM tris-(carboxyethyl)phosphine (TCEP),
h) 10 g/L CDMEAB, 100 mM potassium phosphate, and 50 mM TCEP,
i) 10 g/L CTAC, 100 mM potassium phosphate, and 50 mM TCEP,
j) 10 g/L CTAB, 100 mM potassium phosphate, and 50 mM TCEP,
k) 10 g/L CTAC, 100 mM KCl, and 50 mM TCEP,
l) 10 g/L CTAB, 100 mM KCl, and 50 mM TCEP.

During the sample preparation, proteins were dissolved in the sample denaturing solution and incubated at 90° C. for 2 min. Proteins were also successfully denatured by incubation at 70° C. for 10 min. Some proteins, e.g., lysozyme, were resistant to the thermal denaturation with cationic surfactants and an extended incubation at high temperature (95° C.) was necessary for some formulations (5 min in case of lysozyme). Proteins such as BSA, on the other hand, did not require any denaturation at all prior to electrophoresis.

### Example 3

### The Method of Capillary Sieving Electrophoresis

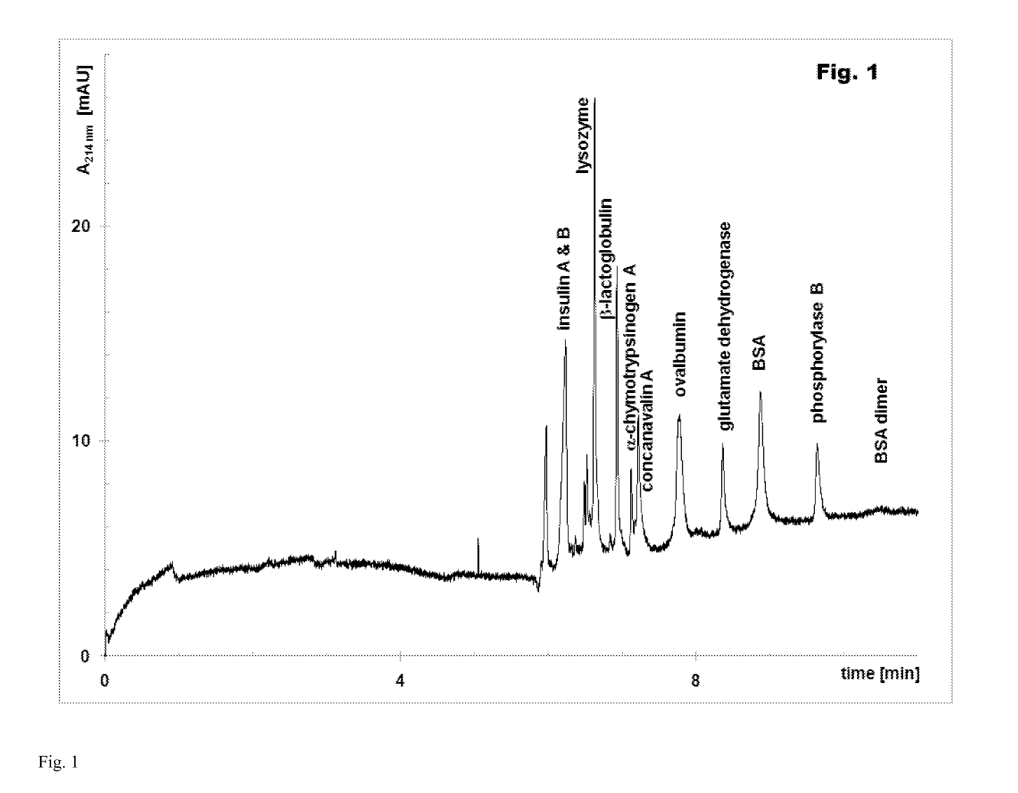

Capillary sieving electrophoresis with a cationic surfactant was performed in bare or coated fused silica capillary (U.S. Pat. No. 7,799,195), 75 μm ID, 360 μm OD, 335 mm total length, 250 mm effective length. In bare capillaries, poly(ethylene oxide) (PEO) was usually a sieving polymer, but for high-resolution separations, capillaries with internal hydrophilic coating were preferred. After each electrophoretic run, the capillary was flushed with 100 mM citric acid at pressure of 930 mbar for 7 min to remove the sieving matrix from the previous run and wash away proteins and other material potentially adsorbed on the capillary wall. In the next step, the capillary was prepared for the next run: the fresh sieving matrix was pumped into the capillary with pressure of 930 mbar for 3 min. The samples were injected either electrokinetically or by pressure. The amount of the injected sample depended on the protein concentration in the sample. The samples prepared with the sample denaturing solution containing 10 g/L CDMEAB, 100 mM KCl, and 10 g/L dithiotreitol and containing 0.1-1 g/L proteins were typically injected for 8 s at 6 kV. The separation was performed at +10 kV and typically took 10-12 minutes. The separation of a model protein mixture is shown in FIG. 1.

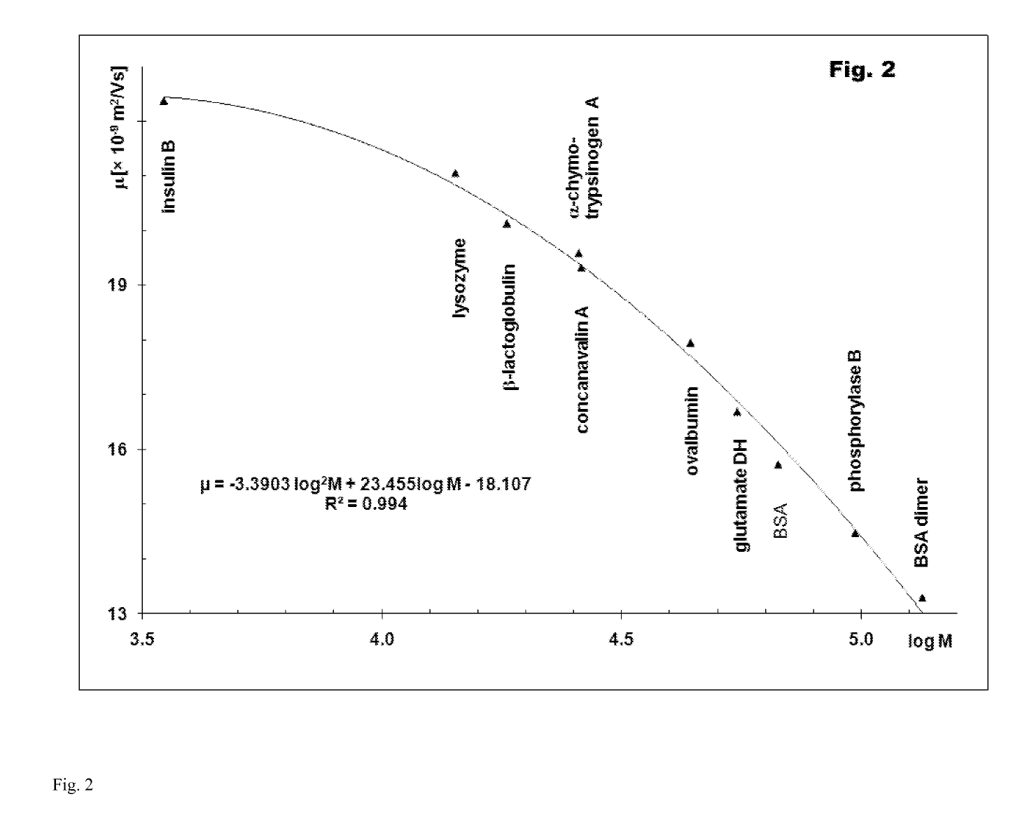

The electrophoretic mobility of the proteins was plotted against the logarithmic molecular weight to provide calibration curve for determination of protein molecular weight (FIG. 2). A quadratic equation was preferred to mathematically express the relationship between electrophoretic mobility and logarithmic molecular weight.

### Example 4

The method of capillary sieving electrophoresis in bare capillary with non coating polymer

Capillary sieving electrophoresis with a cationic surfactant was performed in a bare capillary, 75 μm ID, 360 μM OD, 335 mm total length, 250 mm effective length. For separations in bare capillaries with linear polyacrylamide or dextran as sieving polymers, the bare capillary had to be flushed with a PEO solution before filling with the separation medium.

After each electrophoretic run, the capillary was flushed with 400 mM β-alanine, 400 mM 2-hydroxisobutyric acid. at pressure of 930 mbar for 7 min to remove the sieving matrix from the previous run and wash off proteins and other material potentially adsorbed on the capillary wall. In the next step, the capillary was flushed with 10 g/L poly(ethylene oxide) (M_{w} 7,000,000), 400 mM β-alanine, 400 mM 2-hydroxisobutyric acid to suppress electroosmotic flow. Then a fresh sieving matrix was pumped into the capillary with pressure of 930 mbar for 3 min. The sieving matrix contained 100 mM β-alanine, 100 mM 2-hydroxisobutyric acid, 2 g/L CTAC, and 50 g/L dextran (M_{w} 2,000,000). Samples containing 0.8 g/L model proteins in 100 mM KCl, 10 g/L CTAC, 4 g/L tris-(carboxyethyl)phosphine were electrokinetically injected by applying voltage 8 kV for 15 s.

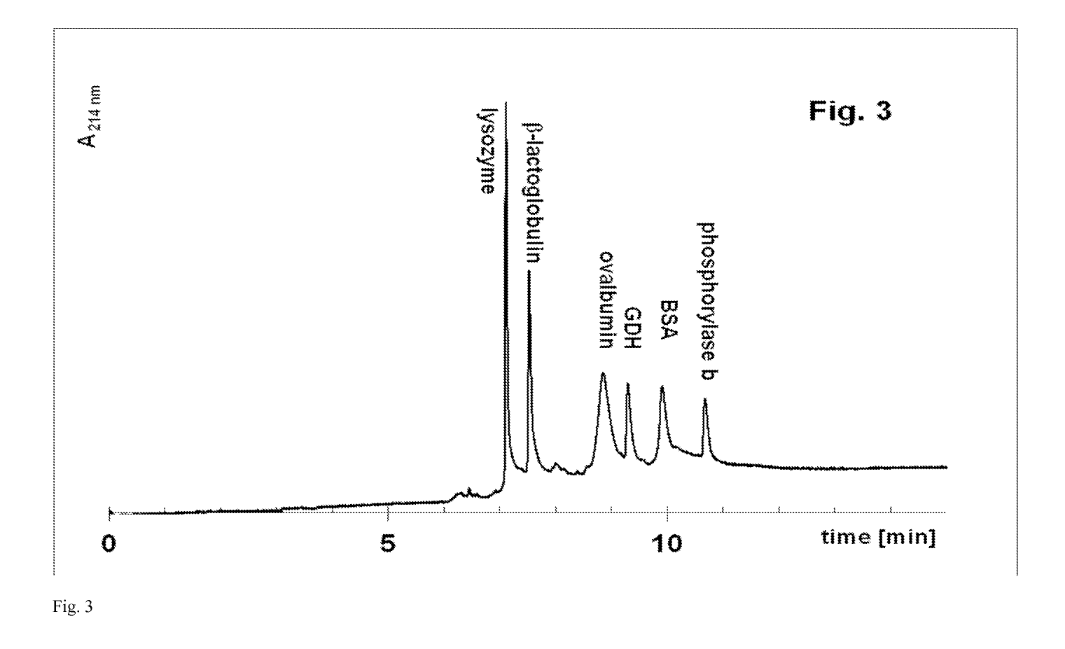

Proteins were separated at voltage of +10 kV and detected by measuring UV absorbance at 214 nm (FIG. 3).

### Example 5

### The Method of Capillary Sieving Electrophoresis with Cationic Surfactant for Separation of Large Proteins

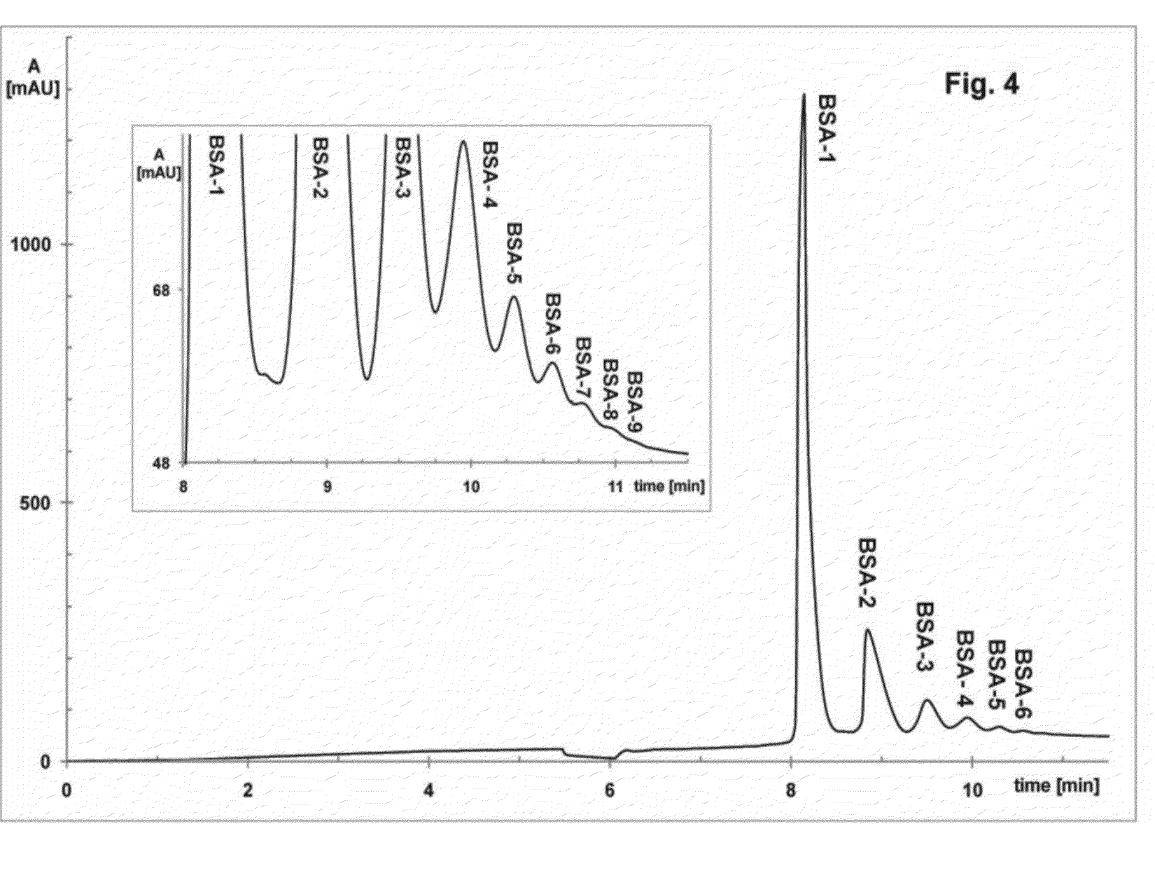

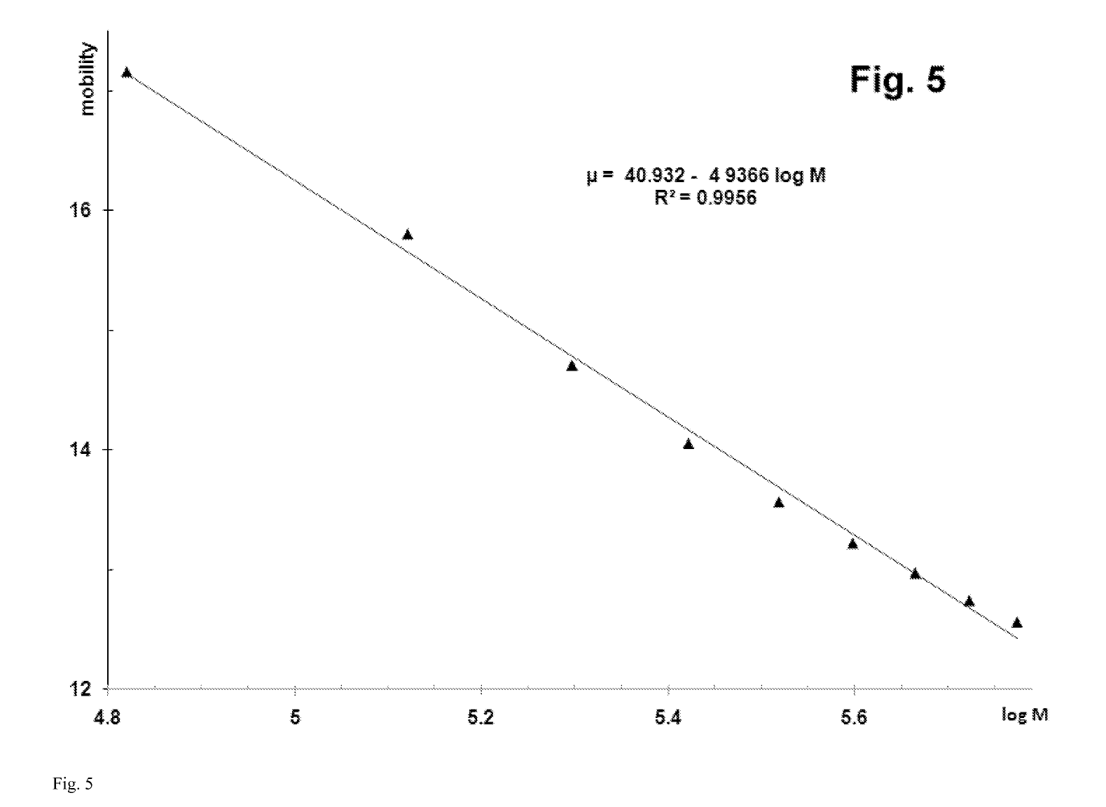

For the separation of native BSA oligomers, the sample containing 10 g/L BSA in 1 g/L CDMEAB was injected at 8 kV for 15 s. The capillary sieving electrophoresis (CSE) of BSA oligomers took about 12 min. and revealed eight to nine peaks (FIG. 4). While the BSA monomer was overloaded, the BSA oligomers from dimer to at least octamer were separated. With the BSA molecular weight of 66,000, it meant proteins were size separated at least in the range between about 14,000 (lysozyme) and about 536,000 (BSA octamer). The electrophoretic mobilities of the BSA oligomers were plotted against the corresponding logarithmic molecular weights (FIG. 5). The obtained straight-line suggested BSA oligomers to be used for calibration of molecular-weight in the range from about 60,000 to about 500,000.

### Example 6

### Separation Efficiency

CSE with a cationic surfactant provided narrow peaks with high separation efficiency. Table 1 summarizes the average separation efficiency of model proteins from 7 runs. The calculation of the separation efficiency from a half-height peak width that assumed ideal Gaussian peaks provided results rather lower than the calculation based on an unrevealed algorithm used in ChemStation software (Agilent).

TABLE 1
Average separation efficiency of protein peaks (n = 7)

| | Average | | | Average | Average | | | Average |
|---|---|---|---|---|---|---|---|---|
| | separation | | | separation | separation | | | separation |
| | efficiency^{a} | | | efficiency^{a} | efficiency^{b} | | | efficiency^{b} |
| | [plates] | SD^{a} | RSD^{a} | [plates/m] | [plates] | SD^{b} | RSD^{b} | [plates/m] |
| insulin B | 52,000 | 3,600 | 7.1% | 208,000 | | | | |
| lysozyme | 445,000 | 46,900 | 10.5% | 1,780,000 | 615,000 | 15,500 | 2.5% | 2,430,000 |
| β-lactoglobulin | 383,000 | 30,400 | 7.9% | 1,532,000 | 526,000 | 18,300 | 3.5% | 2,104,000 |
| α-chymotrypsinogen A | 204,000 | 15,300 | 7.5% | 816,000 | | | | |
| concanavalin A | 111,000 | 22,000 | 19.9% | 444,000 | | | | |
| ovalbumin | 46,000 | 2,700 | 5.9% | 184,000 | | | | |
| glutamate DH | 148,000 | 17,900 | 12.1% | 592,000 | | | | |
| BSA monomer | 54,000 | 12,000 | 22.3% | 216,000 | | | | |
| phosphorylase b | 134,000 | 14,300 | 10.7% | 536,000 | | | | |

^{a}calculated from half-height peak width
^{b}obtained directly from the ChemStation software (Agilent)

### Example 7

### Reproducibility of Migration Times

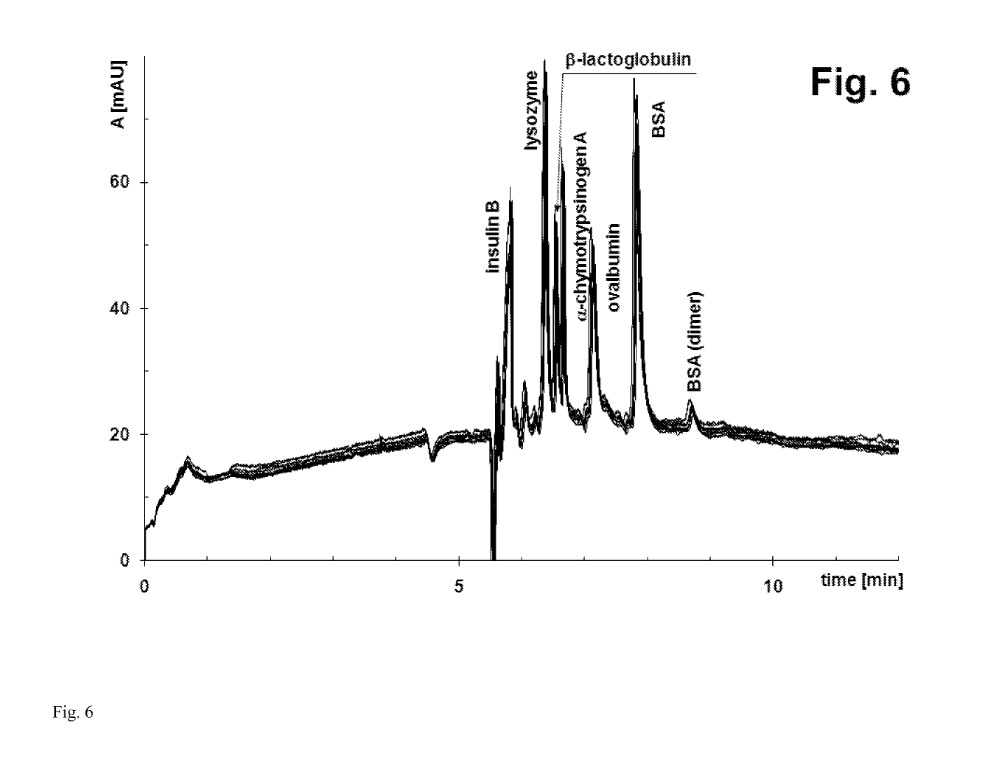

10 overlaid consecutive electropherograms of a model mixture containing 0.8 g/L of insulin B, lysozyme, β-lactoglobulin, α-chymotrypsinogen A, ovalbumin, and BSA are shown in FIG. 6. Run-to-run reproducibility of the migration times ranged from 0.14% to 0.25% and is summarized in Table 2.

TABLE 2
Run-to-run reproducibility of migration times (n = 10).

| | Average tₘ | SD | RSD |
|---|---|---|---|
| Protein | [min] | [min] | [%] |
| Insulin B | 6.09 | 0.009 | 0.14 |
| Lysozyme | 6.68 | 0.010 | 0.15 |
| β-lactoglobulin | 6.88 | 0.010 | 0.15 |
| α-chymotrypsinogen A | 7.04 | 0.011 | 0.16 |
| Ovalbumin | 7.56 | 0.014 | 0.19 |
| BSA | 8.27 | 0.021 | 0.25 |

### Example 8

### Quantitative Analysis

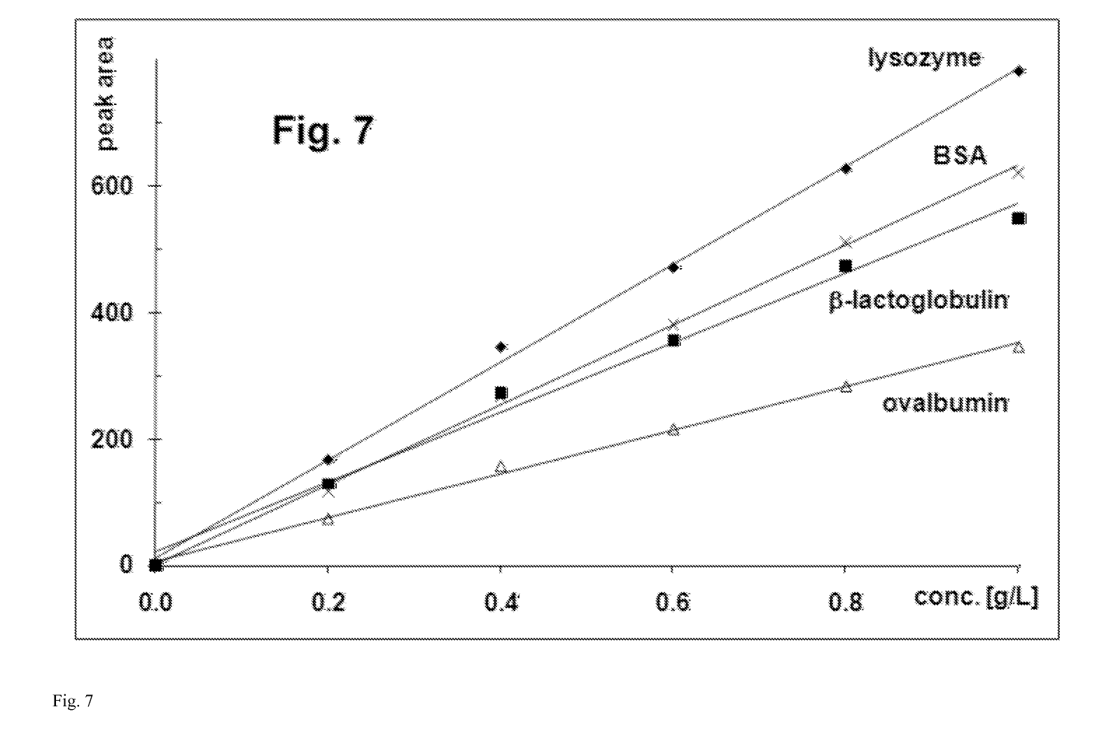

CSE with cationic surfactant allowed quantitative analysis with electrokinetic injection. When proteins were denatured in 10 g/L CDMEAB, 100 mM KCl, and 10 g/L DTT and injected 30 s at +10 kV, the calibration lines for lysozyme, β-lactoglobulin, ovalbumin, and BSA were linear in the concentration range 0-1.0 g/L (FIG. 7). The squared correlation coefficient ranged from 0.99 for β-lactoglobulin to 0.998 for BSA, In terms of the peak area reproducibility, the relative standard deviation ranged from 1.1% (lysozyme) to 2.1% (BSA) at 0.2 g/L (n=10).

TABLE 3
Reproducibility of the peak area for 0.2 g/L proteins
injected 30 s at +10 kV (n = 10).

| | Average | SD | RSD |
|---|---|---|---|
| lysozyme | 168.3 | 1.8 | 1.1% |
| β-lactoglobulin | 133.6 | 1.4 | 1.1% |
| ovalbumin | 76.5 | 1.0 | 1.4% |
| BSA | 121.9 | 2.6 | 2.1% |

What is claimed:

1. A protein denaturing solution for sample preparation prior capillary electrophoretic size separation of proteins, comprising:
from about 1 g/L to about 30 g/L cationic surfactant selected from the group consisting of cetyldimethylethylammonium bromide, cetyltrimethylammonium chloride, cetyltrimethylammonium bromide, tetradecyltrimethylammonium bromide, dodecyltrimethylammonium bromide, cetylamine, tetradecylamine, dodecylamine, decylamine, and didodecyldimethylammonium bromide;
a reducing agent selected from the group consisting of from about 1 g/L to about 30 g/L dithiothreitol, from about 20 mM to about 50 mM tris-(carboxyethyl)phosphine, and from about 1 g/L to about 30 g/L 2-mercaptoethanol; and
from about 20 mM to about 300 mM electrolyte selected from the group consisting of potassium chloride, potassium phosphate, and ammonium acetate.

2. The protein denaturing solution for sample preparation prior capillary electrophoretic size separation of proteins of claim 1, wherein said protein denaturing solution comprises about 10 g/L cetyldimethylethylammonium bromide, about 100 mM potassium phosphate, and about 10 g/L dithiothreitol.

3. The protein denaturing solution for sample preparation of proteins prior capillary electrophoretic size separation of claim 1, wherein said protein denaturing solution comprises about 10 g/L cetyldimethylethylammonium bromide, about 100 mM potassium chloride, and about 10 g/L dithiothreitol.

4. The protein denaturing solution for sample preparation of proteins prior capillary electrophoretic size separation of claim 1, wherein said protein denaturing solution comprises about 10 g/L cetyltrimethylammonium chloride, about 100 mM potassium phosphate, and about 10 g/L dithiothreitol.

5. The protein denaturing solution for sample preparation of proteins prior capillary electrophoretic size separation of claim 1, wherein said protein denaturing solution comprises about 10 g/L cetyltrimethylammonium chloride, about 100 mM potassium phosphate, and from about 20 mM to about 50 mM tris-(carboxyethyl)phosphine.

6. The protein denaturing solution for sample preparation of proteins prior capillary electrophoretic size separation of claim 1, wherein said protein denaturing solution comprises about 10 g/L cetyltrimethylammonium bromide, about 100 mM potassium chloride, and from about 20 mM to about 50 mM tris-(carboxyethyl)phosphine.

7. A method of sample preparation prior capillary electrophoretic size separation of proteins, wherein a protein sample is dissolved in the protein denaturation solution of claim 1 and incubated at temperature from about 60 to about 100° C. for a time interval from about 1 min to about 20 min.

8. A method of sample preparation prior capillary electrophoretic size separation of proteins of claim 7, wherein a protein sample is dissolved in the protein denaturation solution of claim 1 and incubated at about 90° C. for about 2 min.

9. A method of sample preparation prior capillary electrophoretic size separation of proteins of claim 7, wherein a protein sample is dissolved in the protein denaturation solution of claim 1 and incubated at about 70° C. for about 10 min.

10. A separation medium for capillary electrophoretic size separation of proteins, consisting essentially of:
a cationic surfactant, selected from the group of surfactants consisting of:
cetyldimethylethylammonium bromide, tetradecyltrimethylammonium bromide, dodecyltrimethylammonium bromide, cetylamine, tetradecylamine, dodecylamine, decylamine, and didodecyldimethylammonium bromide.
an acidic buffer having pH in the range from about 3 to about 5.5, wherein said acidic buffer comprises at least one of the following buffering substances selected from the group consisting of:
glycine, β-alanine, γ-aminobutyric acid, δ-aminovaleric acid, ε-aminocaproic acid, nicotinamide, formate, acetate, propionate, lactate, 2-hydroxybutyrate, 2-hydroxyisobutyrate, nicotinate, glutamate, and aspartate; and
a sieving polymer, wherein said sieving polymer is selected from the group consisting of:
linear polyacrylamide, poly(dimethyl acrylamide), poly(hydroxyethyl acrylamide), poly(hydroxypropyl acrylamide), poly(vinyl alcohol), poly(vinyl pyrrolidone), hydroxyethyl cellulose, guaran, locust bean gum, glucomannan, pullulan, and dextran.

11. The separation medium for capillary electrophoretic size separation of proteins of claim 10, wherein said cationic surfactant is cetyldimethylethylammonium bromide in the concentration range from about 0.5 g/L to about 30 g/L.

12. The separation medium for capillary electrophoretic size separation of proteins of claim 10, wherein said cationic surfactant is tetradecyltrimethylammonium bromide in the concentration range from about 0.5 g/L to about 30 g/L.

13. The separation medium for capillary electrophoretic size separation of proteins of claim 10, wherein said acidic buffer comprises from about 20 mM to about 200 mM β-alanine, and from about 20 mM to about 200 mM glutamic acid.

14. The separation medium for capillary electrophoretic size separation of proteins of claim 10, wherein said acidic buffer comprises from about 20 mM to about 200 mM γ-aminobutyric acid, and from about 20 mM to about 200 mM glutamic acid.

15. The separation medium for capillary electrophoretic size separation of proteins of claim 10, wherein said acidic buffer comprises from about 20 mM to about 200 mM β-alanine and from about 20 mM to about 200 mM 2-hydroxyisobutyric acid.

16. The separation medium for capillary electrophoretic size separation of proteins of claim 10, consisting essentially of:
from about 8 g/L to about 30 g/L polyacrylamide, having molecular weight from about 100,000 to about 1,000,000;
from about 20 mM to about 200 mM γ-aminobutyric acid;
from about 20 mM to about 200 mM glutamic acid; and
from about 0.5 g/L to about 30 g/L cetyldimethylethylammonium bromide.

17. A separation medium for capillary electrophoretic size separation of proteins of claim 10, consisting essentially of 15 g/L polyacrylamide having molecular weight 600,000-1 million, 100 mM β-alanine, 100 mM glutamic acid, and 1 g/L cetyldimethylethylammonium bromide.

18. The separation medium for capillary electrophoretic size separation of proteins of claim 10, consisting essentially of:
from about 60 g/L to about 120 g/L dextran, having molecular weight about 2,000,000;
from about 20 mM to about 200 mM γ-aminobutyric acid;
from about 20 mM to about 200 mM glutamic acid; and
from about 0.5 g/L to about 30 g/L cetyldimethylethylammonium bromide.

19. A separation medium for capillary electrophoretic size separation of proteins of claim 10, consisting essentially of 100 g/L dextran having molecular weight 2,000,000, 100 mM γ-aminobutyric acid, 100 mM glutamic acid, and 2 g/L cetyldimethylethylammonium chloride.

20. A method for capillary electrophoretic size separation of proteins, comprising the following steps:
a) sample preparation of claim 7 wherein a protein sample is dissolved in said protein denaturation solution of claim 1 and incubated at temperature from about 60 to about 100° C. for an interval from about 1 min to about 20 min;
b) flushing the separation capillary with from about 5 g/L to about 30 g/L polyethylene oxide to form a dynamic coating;
c) filling the separation capillary with said separation medium for capillary electrophoretic size separation of proteins of claim 10;
d) sample injection, with the injection voltage from about 0.5 kV to about 12 kV applied for from about 1 s to about 60 s;
e) separation, with the separation voltage from about 1 kV to about 20 kV applied for about 5 minute to about 30 minutes;
f) detection, wherein the absorption of monochromatic light having wavelength from about 210 nm to about 220 nm is measured and plotted in an electropherogram.

21. Said method for capillary electrophoretic size separation of proteins of claim 20, wherein said sieving polymer in said separation medium for capillary electrophoretic size separation of proteins of claim 10 is about 100 g/L dextran with molecular weight of about 2 million.

22. Said method for capillary electrophoretic size separation of proteins of claim 20, wherein said sieving polymer in said separation medium for capillary electrophoretic size separation of proteins of claim 10 is from about 8 g/L to about 30 g/L polyacrylamide, having molecular weight from about 100,000 to about 1,000,000.
